(19) United States
(12) Patent Application Publication
(10) Pub. No.: US 2005/0129764 A1
(43) Pub. Date: Jun. 16, 2005

(54) Osmotic device containing licofelone

(76) Inventors: Juan A. Vergez, Buenos Aires (AR); Joaquina Faour, Buenos Aires (AR); Marcelo A. Ricci, Buenos Aires (AR); Marcelo E. Befumo, Buenos Aires (AR)

Correspondence Address: INNOVAR, LLC, P O BOX 250647, PLANO, TX, 75025, US

(21) Appl. No.: 10/733,156

(22) Filed: Dec. 11, 2003

(51) Int. Cl. A61K 31/415; A61K 9/24

(52) U.S. Cl. 424/473; 514/406

(57) ABSTRACT

The present invention provides an osmotic device containing controlled release licofelone and, optionally, a rapid release licofelone in an external coat. The osmotic devices provide a controlled release of licofelone to maintain therapeutically effective levels of licofelone in plasma when administered once per day. The device is useful for the treatment of osteoarthritis, rheumatoid arthritis, and inflammatory related disorders. The present devices provide licofelone according to specific release profiles in combination with specific formulations.

### FIELD OF THE INVENTION

This invention pertains to an osmotic device containing licofelone. More particularly, it pertains to an osmotic device tablet, which provides a controlled release of licofelone to maintain therapeutically effective levels of licofelone in plasma when administered once per day.

### BACKGROUND OF THE INVENTION

Licofelone (ML-3000) is a pyrrolizine derivative orally-active dual cyclooxygenase-1 and -2 and 5-lipoxygenase inhibitor (dual acting anti-inflammatory drug), under development as an anti-inflammatory and analgesic by the EuroAlliance consortium (Alfa Wassermann/Lacer/Merckle). Licofelone is undergoing evaluation in clinical trials for the indication of osteoarthritis (Laufer S., Expert Opin Investig Drugs 12(7):1239-41, 2003; Reginster J. et al., Annual European Congress of Rheumatology, EULAR 2002, p.abstr. THU0189, 12 Jun. 2002), rheumatoid arthritis (Gay R. E., et al., J Rheumatol 28(9):2060-5, 2001) and pain. In animal experiments the compound has antiphlogistic, analgesic, antipyretic, antiasthmatic and antiaggregative activity at a dosage that causes no gastrointestinal damage (Laufer S. Et al, Arzneimittelforschung 44(5):629-36), 1994). Results of a phase III trial showed that licofelone was at least as effective as naproxen in the long-term treatment of osteoarthritis of the knee (n=704). At dosages of licofelone 200 and 400 mg/day bid and naproxen 1000 mg/day bid, the greatest improvement in efficacy parameters was achieved with licofelone 400 mg/day bid (Blanco F. J. Et al, Ann Rheum Dis 62(1):262, 2003).

In accordance with the prior art, licofelone doses of 200 and 400 mg/day, administered as divided doses have been found effective for many patients. Following a 200 mg licofelone dose administered twice daily, peak plasma licofelone concentrations of about (1650-1750) ng/ml were reached at (0.74-4) h post-dose. Licofelone exhibited an elimination half-life (T_{1/2β}) of about (8.7-11.1) hours (Albrecht W. et al., Annual European Congress of Rheumatology, EULAR 2002, p.abstr. AB0293 12 Jun. 2002). A single dose of licofelone (800-3,200) mg (4-fold the therapeutic dose) was generally well tolerated in volunteers (Bias P. Et al., Ann Rheum Dis 62 (1): 479 2003).

Osmotic devices and other tablet formulations are known for their ability to provide a controlled release of a wide range of drugs. Such osmotic devices and other tablet formulations are disclosed in U.S. Pat. No. 4,014,334 to Theeuwes et al., U.S. Pat. No. 4,576,604 to Guittard et al., Argentina Patent No. 234,493, U.S. Pat. No. 4,673,405 to Guittard et al., U.S. Pat. No. 5,558,879 to Chen et al., U.S. Pat. No. 4,810,502 to Ayer et al., U.S. Pat. No. 4,801,461 to Hamel et al., U.S. Pat. No. 5,681,584 to Savastano et al., U.S. Pat. No. 3,845,770, U.S. Pat. No. 4,008,719 to Theeuwes et al., U.S. Pat. No. 4,058,122 to Theeuwes et al., U.S. Pat. No. 4,116,241 to Theeuwes et al., U.S. Pat. No. 4,160,452 to Theeuwes, U.S. Pat. No. 4,256,108 to Theeuwes, and Argentina Patent No. 199,301, the entire disclosures of which are hereby incorporated by reference. Osmotic devices such as those described by Faour et al. (U.S. Pat. No. 6,004,582), the entire disclosure of which is hereby incorporated by reference, are particularly advantageous for delivering two different dosages from a single osmotic device tablet. The prior art does not disclose the administration of licofelone with a prolonged release dosage form nor the administration of licofelone with an osmotic device.

While controlled release dosage forms, such as described above, are effective for the controlled release of many different pharmaceutical agents known in the art, however, they do not disclose osmotic devices that provide the specific formulations, plasma profiles or release profiles for licofelone claimed herein. It would be an advance in the art to provide patients with effective plasma licofelone concentrations while providing relatively lower peak plasma licofelone concentrations within each dosing interval. In this manner, the potential for peak-associated problems is minimized. Moreover, a once-a-day dosing schedule results in a simpler and more convenient drug therapy regimen that may improve patient compliance.

### SUMMARY OF THE INVENTION

In one embodiment, the present invention provides an osmotic device comprising:

a core comprising a therapeutically effective amount of licofelone and at least one osmotic agent or osmopolymer; and

a semipermeable membrane surrounding the core and having a passageway there through;

wherein at least 75% of licofelone is released within 24 hours.

In another embodiment, the present invention provides an osmotic device comprising:

a core comprising a first amount of licofelone and at least one osmotic agent or osmopolymer;

a semipermeable membrane surrounding the core and having a passageway there through; and

an external coat comprising a second amount of licofelone;

wherein at least 75% of licofelone is released within 24 hours, and the first and second amounts of licofelone together comprise a therapeutically effective amount of licofelone.

Yet another embodiment of the present invention provides an osmotic device comprising:

a core comprising a therapeutically effective amount licofelone and at least one osmotic agent or osmopolymer, wherein the core provides a controlled release of licofelone;

a semipermeable membrane surrounding the core and having a passageway there through;

an inert erodible and/or water soluble lamina plugging the preformed passageway and at least partially surrounding the semipermeable membrane; and

a drug-containing external coat surrounding the inert lamina, the coat comprising a therapeutically effective amount of licofelone, wherein at least 75% of licofelone is released within 24 hours.

In other embodiments, the external coat is applied by spray coating rather than by compression coating. By spray coating rather than compression coating the external coat is thinner, and therefore a smaller osmotic device is formed.

Other embodiments include those wherein: 1) at least 75% of the licofelone contained in the external coat is released within 30 min after administration; 2) at least 75% of the licofelone contained in the external coat is released in 20 min after administration; 3) at least 75% of the licofelone contained in the external coat is released within 10 min after administration; 4) at least 75% of the licofelone contained in the external coat is released within 5 min after administration; 5) all of the licofelone contained in the external coat is released within 90 min after administration; 6) all of the licofelone contained in the external coat is released within 45 min after administration; 7) all of the licofelone contained in the external coat is released within 30 min after administration; 8) all of the licofelone contained in the external coat is released within 20 min after administration; 9) all of the licofelone contained in the external coat is released within 10 min after administration; 10) all of the licofelone contained in the external coat is released within 5 min after administration; 11) the osmotic device further comprises an inert and erodible water soluble lamina interposed the semipermeable membrane and the drug-containing outer coating; 12) the water soluble lamina comprises poly(vinylpyrrolidone)-(vinyl acetate) copolymer; 13) all of the licofelone contained in the external coat is released within 120 min after administration; 14) all of the licofelone contained in the external coat is released within 180 min after exposure to an aqueous environment, 15) after 1 hour of exposure to an aqueous environment 20-30% of the licofelone is released; 16) after 4 hours of exposure to an aqueous environment 25-65% of the licofelone is released; 17) after 12 hours of exposure to an aqueous environment 47-83% of the licofelone is released; 18) after 24 hours of exposure to an aqueous environment not less than 75% of the licofelone is released; 19) at least 20% of the licofelone is released within 4 hours of exposure to an aqueous environment; 20) at least 45% of the licofelone is released within 12 hours of exposure to an aqueous environment; 21) at least 60% of the licofelone is released within 16 hours of exposure to an aqueous environment; and/or 22) at least 75% of the licofelone is released within 20 hours of exposure to an aqueous environment.

Yet other embodiments includes those wherein the licofelone is released at a zero order or pseudo-zero order rate for a period of at least 12 hours, at least 14 hours, at least 16 hours, at least 18 hours, and at least 20 hours.

In other embodiments, the licofelone release profile resembles a sigmoid such that release of licofelone occurs more slowly initially, then accelerates after a first period of time and finally decelerates after a second period of time.

Another embodiment of the invention provides a method of treating a condition in a subject, the condition being responsive to licofelone, the method comprises the step of administering an osmotic device, which provides a controlled release of licofelone from its core and, optionally, a rapid release of licofelone from an external coat, wherein at least 75% of the licofelone is released is released within about 24 hours.

In other embodiments, the osmotic device has a licofelone release profile similar to that shown in FIGS. 1, 2, 5, or 6. In yet another embodiment, the plasma levels for licofelone are similar to that shown in FIGS. 3 or 4.

In one embodiment, the controlled release osmotic device of the invention releases licofelone at a release rate that results in lower peak plasma licofelone concentrations following dose administration than are obtained following administration of an immediate-release licofelone dosage form containing the same dose of licofelone.

Another embodiment of the invention provides a method of treating a condition or disorder responsive to licofelone, the method comprising the step of administering a prolonged release dosage form comprising a therapeutically effective amount of licofelone, wherein at least 75% of the licofelone is released substantially continually over a period of at least 12 hours.

Other features, advantages and embodiments of the invention will become apparent to those skilled in the art by the following description, accompanying examples.

### BRIEF DESCRIPTION OF THE FIGURES

The following drawings are part of the present specification and are included to further demonstrate certain aspects of the invention. The invention may be better understood by reference to one or more of these drawings in combination with the detailed description of the specific embodiments presented herein.

FIG. 1 depicts an in vitro dissolution profile of licofelone released from the exemplary formulation of Example 1.

FIG. 2 depicts an in vitro dissolution profile of licofelone released from the exemplary formulation of Example 2.

FIG. 3 depicts plasma licofelone concentration profiles following the administration of a single 200 mg dose of licofelone in an immediate release dosage form vs. a single dose of 400 mg of licofelone osmotic device of Example 1

FIG. 4 depicts a simulated plasma licofelone concentration profile following dosing every 24 hours with 400 mg doses of licofelone osmotic device of Example 1, vs. dosing every 12 hours with 200 mg doses of licofelone (total daily dose=400 mg) in an immediate release dosage form.

FIG. 5 depicts a chart of licofelone release profiles for a prolonged release dosage form of the invention.

FIG. 6 depicts a chart of licofelone release profiles for a prolonged release dosage form of the invention, wherein licofelone is released rapidly and then according to a prolonged release profile.

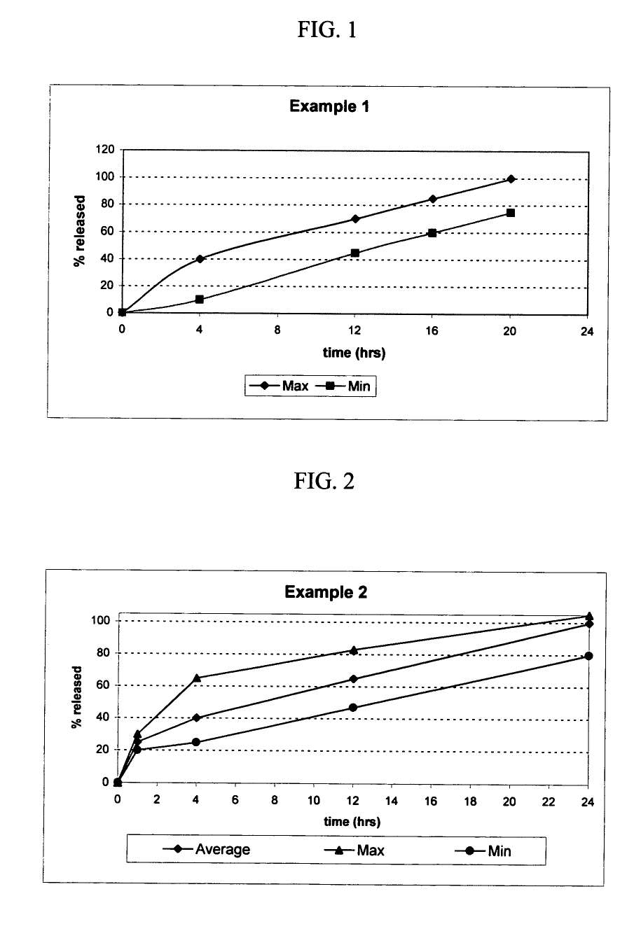

### DETAILED DESCRIPTION OF THE INVENTION

The invention provides for the administration of licofelone in its free acid, racemic, optically pure, stereoisomeric and/or pharmaceutically acceptable salt forms. The invention also provides for the administration of derivatives and analogues of licofelone. Possible routes for preparation of licofelone are described in U.S. Pat. No. 6,417,371, U.S. Pat. No. 5,958,943 and U.S. Pat. No. 5,942,535, EP0397175, Archiv der Pharmazie 312:896-907 (1979); and 321:159-162 (1988), and Arch. Pharm. Pharm. Med. Chem. 330:307-312 (1997).

“Pharmaceutically acceptable salts” refer to derivatives of the disclosed compounds wherein the therapeutic compound is modified by making acid or base salts thereof. Examples of pharmaceutically acceptable salts include, but are not limited to, mineral or organic acid salts of the licofelone. The pharmaceutically acceptable salts include the conventional non-toxic salts, for example, from non-toxic inorganic or organic acids. For example, such conventional non-toxic salts include those derived from inorganic acids such as hydrochloric, hydrobromic, sulfuric, sulfonic, sulfamic, phosphoric, nitric and the like; and the salts prepared from organic acids such as amino acids, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and other known to those of ordinary skill in the pharmaceutical sciences. Lists of suitable salts are found in texts such as Remington's Pharmaceutical Sciences, 18^{th} Ed. (Alfonso R. Gennaro, ed.; Mack Publishing Company, Easton, Pa., 1990); Remington: the Science and Practice of Pharmacy 19^{th} Ed.( Lippincott, Williams & Wilkins, 1995); Handbook of Pharmaceutical Excipients, 3^{rd} Ed. (Arthur H. Kibbe, ed.; Amer. Pharmaceutical Assoc., 1999); the Pharmaceutical Codex: Principles and Practice of Pharmaceutics 12^{th} Ed. (Walter Lund ed.; Pharmaceutical Press, London, 1994); The United States Pharmacopeia: The National Formulary (United States Pharmacopeial Convention); and Goodman and Gilman's: the Pharmacological Basis of Therapeutics (Louis S. Goodman and Lee E. Limbird, eds.; McGraw Hill, 1992), the disclosures of which are hereby incorporated by reference.

“Pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A “therapeutically effective amount” is the amount or quantity of drug, which is sufficient to elicit the required or desired therapeutic response, or in other words, the amount which is sufficient to elicit an appreciable biological response when administered to a patient.

An immediate release dosage form is one that begins to release drug shortly, generally seconds to minutes, after exposure to an environment of use and therefore does not exhibit a significant delay in release of drug.

A rapid release dosage form is one that releases drug over a period of 1-59 minutes or 0.1 to three hours once release has begun.

As used herein, a prolonged release dosage form is one that releases licofelone over a prolonged period of time. The release is substantially continuous and occurs over at least a minimum period of time. For example, release might occur over a period of at least 8, 10, 12, 16, 18, 20, 22 or 24 hours as measured from initial exposure of the dosage form to an aqueous environment of use. A prolonged release dosage form may provide a controlled release, sustained release or extended release of licofelone. A prolonged release dosage form can be adapted to release drug at a substantially constant rate or to release a substantially constant incremental amount of drug over a prolonged period of time.

By “controlled release” is meant release of an active agent to an environment of use over a period of about 3 hours up to about 12 hours, 16 hours, 18 hours, 20 hours, a day, or more than a day.

By “sustained release” is meant controlled release of an active agent in a manner such that the dosage form maintains a constant drug level in the blood or target tissue of a subject to which the dosage form is administered.

By “extended release” is meant controlled release of an active agent from a dosage form to an environment of use in a manner to allow at least a two-fold reduction in frequent dosing compared to the drug presented in a conventional dosage form (e.g., a solution or rapid releasing conventional solid dosage forms containing the same active agent).

A “zero-order” release profile characterizes the release profile of a dosage form that releases a constant amount of drug per unit time. A “pseudo-zero order” release profile is one that approximates a zero-order release profile.

A “first order” release profile characterizes the release profile of a dosage form that releases a constant percentage of an initial drug charge per unit time. A “pseudo-first order” release profile is one that approximates a first order release profile.

Licofelone may be provided in the osmotic device of the present invention in amount of 200 mg to up 800 mg or more if desired. In specific embodiments of once-a-day dosage forms in agreement with the present invention, the osmotic device provides a controlled release of licofelone in doses of 200, 400 and 800 mg of licofelone per dosage form. Three different embodiments of such dosage forms are exemplified in Example 1. In other embodiments, the once a day dosage forms comprise 150, 300, and 600 mg of controlled release licofelone in combination with 50, 100, and 200 mg of immediate or rapid release licofelone, respectively, per dosage form as exemplified in Example 2.

The release profile of the osmotic device of the invention will vary according to the materials used to form the core and the semipermeable membrane covering the core. For example, the release profile can be influenced by the material used to form the semipermeable membrane covering the core, by the material used to form any coating on the semipermeable membrane, by the excipients present in the core, or by the presence of an osmagent in the core. The release profiles for the formulations of Example 1 are generally described as follows:

| | Maximum Percent | Minimum Percent |
|---|---|---|
| Time (h) | Released | Released |
| 1 | 15 | 0 |
| 4 | 40 | 10 |
| 12 | 70 | 45 |
| 16 | 85 | 60 |
| 20 | 101 | 75 |

In standard dissolution assays, the values can vary depending upon the conditions employed. Moreover, the values may have an absolute standard deviation (STD) of ±10%, ±5% or ±3% at each given time point.

The tablets of the invention will generally provide therapeutically effective amounts of licofelone for a period of not less than 18 hours and not more than 30 hours, not less than 20 hours and not more than 28 hours, not less than 18 hours and not more than 24 hours, or not less than 22 hours and not more than 24 hours.

The formulations of Example 2 provide licofelone dissolution profiles as depicted in FIG. 2. The licofelone release profiles of FIG. 2 are generally described as follows:

| | | | |
|---|---|---|---|
| Time | Average | STD | Range (%) |

| (h) | (%) | (%) | Max | Min |
|---|---|---|---|---|
| 1 | 25 | 10.2 | 30 | 20 |
| 4 | 40 | 7.4 | 65 | 25 |
| 12 | 65 | 6.4 | 83 | 47 |
| 24 | 100 | 5.5 | 105 | 80 |

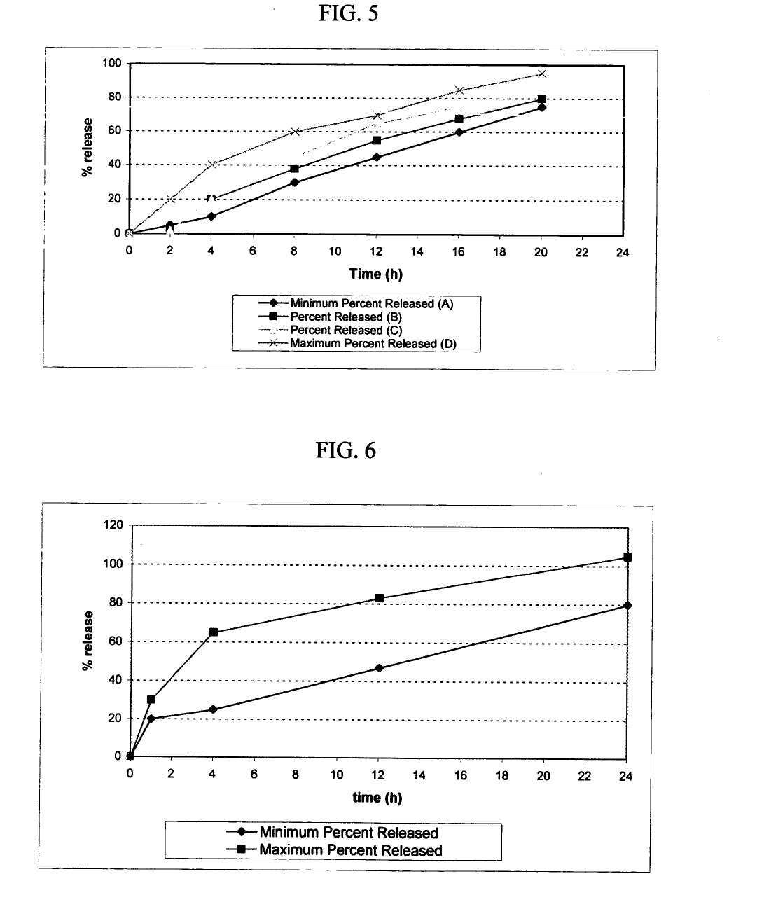

Exemplary release profiles for a prolonged release dosage form comprising licofelone are detailed in the following table and as depicted in FIG. 5 (curves A and D).

| Time | Minimum Percent | Maximum Percent |
|---|---|---|
| (h) | Released (A) | Released (D) |
| 0 | 0 | 0 |
| 1 | 2 | 10 |
| 2 | 5 | 20 |
| 4 | 10 | 40 |
| 8 | 30 | 60 |
| 12 | 45 | 70 |
| 16 | 60 | 85 |
| 20 | 75 | 95 |

The above noted release profile is suitable for zero order release, pseudo zero order release, first order release, pseudo first order release, delayed and then zero (or pseudo zero) order release, or delayed and then first (or pseudo first) order release.

Other exemplary release profiles can be characterized as follows and as depicted in FIG. 5 (curvesB and C).

| Time | Percent Released | Percent Released |
|---|---|---|
| (h) | (B) | (C) |
| 0 | 0 | 0 |
| 1 | 1 | 1 |
| 2 | 2 | 2 |
| 4 | 20 | 20 |
| 8 | 38 | 45 |
| 12 | 55 | 65 |
| 16 | 68 | 75 |
| 20 | 80 | 85 |

When the external drug-containing coating is present, there is an initial rapid release of licofelone followed by a prolonged release. An exemplary overall (combination of rapid and prolonged release) release profile of a dosage form containing such an external drug-containing coating is depicted in FIG. 6 and is generally described in the table below.

| Time | Minimum Percent | Maximum Percent |
|---|---|---|
| (h) | Released | Released |
| 0 | 0 | 0 |
| 1 | 20 | 30 |
| 4 | 25 | 65 |
| 12 | 47 | 83 |
| 24 | 80 | 105 |

The external coating can be an immediately dissolving coating that dissolves in the buccal cavity or a rapidly dissolving coating that dissolves in the stomach, jejunum or duodenum. The controlled release core generally begins to release licofelone within about 0.5-3 hours or 0.5-2 hours after administration or within less than about 1 hour after administration.

The rapid release external coating will release all of the licofelone within three hours after administration and at least 75% of its licofelone within about 40 minutes after administration.

Those of ordinary skill in the art will appreciate that the particular amounts of licofelone used in the osmotic device will vary according to, among other things, the desired pharmacokinetic behavior in a mammal.

The pharmacokinetics of licofelone dosage forms in accord with the present invention and conventional immediate release dosage forms were compared in a randomized open-label, single dose, two-way crossover study in 12 healthy male and female subjects (Example 3). The reference treatment consisted of a single 200 mg dose of licofelone formulated as a conventional immediate release dosage form containing cellulose microcrystalline, povidone, lactose, colloidal silicon dioxide, and magnesium stearate. The exemplary osmotic device (according to Example 1) comprised a single dose of licofelone (400 mg). The mean Cₘₐₓ (ng/ml) values were 1568.8 for the immediate release dosage form and 520.8 for the osmotic device. The mean Tₘₐₓ (h) following administration of the immediate-release dosage form was just 1.4 hours, while following administration of the osmotic device the mean Tₘₐₓ value was 7.3 hours. Contrary to the immediate-release licofelone dosage form, which must be administered two times per day i.e, every 12 hours, to provide average steady-state plasma licofelone concentrations sufficient for therapeutic effectiveness and which releases the drug in a rapid manner resulting in relatively high peak plasma drug concentrations following each dose, the osmotic device of the invention provides patients with therapeutically effective plasma concentrations of licofelone while providing relatively lower peak plasma concentrations of licofelone within each dosing interval. In this manner, the potential for peak-associated problems is minimized.

In a simulation of mean steady-state plasma licofelone concentrations following dosing every 24 hours with 400 mg doses of licofelone osmotic device of Example 1, and following dosing every 12 hours with 200 mg doses of licofelone (total daily dose=400 mg) in an immediate release dosage form for a period of four days (Example 3), the peak plasma licofelone concentrations are lower following administration of the sustained release dosage form than those observed following administration of the immediate release dosage form. Additionally, the number of peak plasma licofelone concentrations occurring over the four day period with the sustained release dosage form regimens are half the number occurring with the immediate release dosage form regimen, i.e., 4 vs 8 (FIG. 4). As a result of the plasma profile provided by the osmotic device of the invention, a more advantageous method of treatment is provided. The severity and/or frequency of side effects that are plasma concentration dependent can be reduced by use of a prolonged release dosage form of the invention.

The invention provides a method for the treatment of osteoarthritis, rheumatoid arthritis, and related inflammatory diseases. The method is practiced by administering one osmotic device of the invention daily.

A water soluble and/or erodible coating, inert or drug-containing, will generally comprise an inert and non-toxic material which is at least partially, and optionally substantially completely, soluble or erodible in an environment of use. Selection of materials suitable for the inert or drug-containing water soluble coatings will depend upon the desired release rate of drug from the drug-containing coating and upon the desired separation of drug delivery from the core versus the drug-containing coating. A rapidly dissolving coat will be soluble in the buccal cavity and/or upper GI tract, such as the stomach, duodenum, jejunum or upper small intestines. Exemplary materials are disclosed in U.S. Pat. No. 4,576,604 to Guittard et al. and U.S. Pat. No. 4,673,405 to Guittard et al., and U.S. Pat. No. 6,004,582 to Faour et al. and the text Pharmaceutical Dosage Forms: Tablets Volume I, 2^{nd} Edition. (A. Lieberman. ed. 1989, Marcel Dekker, Inc.), the relevant disclosures of which are hereby incorporated by reference. In some embodiments, the rapidly dissolving coat will be soluble in saliva, gastric juices, or acidic fluids.

Materials which are suitable for making the water soluble and/or erodible coatings of the invention include, by way of example and without limitation, water soluble polysaccharide gums such as carrageenan, fucoidan, gum ghatti, tragacanth, arabinogalactan, pectin, and xanthan; water-soluble salts of polysaccharide gums such as sodium alginate, sodium tragacanthin, and sodium gum ghattate; water-soluble hydroxyalkylcellulose wherein the alkyl member is straight or branched of 1 to 7 carbons such as hydroxymethylcellulose, hydroxyethylcellulose, and hydroxypropylcellulose; synthetic water-soluble cellulose-based lamina formers such as methyl cellulose and its hydroxyalkyl methylcellulose cellulose derivatives such as a member selected from the group consisting of hydroxyethyl methylcellulose, hydroxypropyl methylcellulose, and hydroxybutyl methylcellulose; croscarmellose sodium; other cellulose polymers such as sodium carboxymethylcellulose; and other materials known to those of ordinary skill in the art. Other lamina forming materials that can be used for this purpose include poly(vinylpyrrolidone), polyvinylalcohol, polyethylene oxide, a blend of gelatin and polyvinyl-pyrrolidone, gelatin, glucose, saccharides, povidone, copovidone, poly(vinylpyrrolidone)-poly(vinyl acetate) copolymer. The coating can comprise other pharmaceutical excipients that do or do not alter the way in which the water soluble coating behaves. The artisan of ordinary skill will recognize that the above-noted materials include film forming polymers.

Other materials which can be used in the water soluble and/or erodible coatings include hydroxypropylcellulose, microcrystalline cellulose (MCC, Avicel.TM. from FMC Corp.), poly(ethylene-vinyl acetate) (60:40) copolymer (EVAC from Aldrich Chemical Co.), 2-hydroxyethylmethacrylate (HEMA), MMA, terpolymers of HEMA:MMA:MA synthesized in the presence of N,N′-bis(methacryloyloxyethyloxycarbonylamino)-azobenzene, azopolymers, enteric coated timed release system (Time Clock® from Pharmaceutical Profiles, Ltd., UK) and calcium pectinate can be included in the coat.

The inert water soluble and/or erodible coat covering the semipermeable wall and blocking the passageway is made of synthetic or natural material which, through selective dissolution or erosion shall allow the passageway to be unblocked thus allowing the process of osmotic delivery to start. This slow or fast dissolving water soluble coat can be impermeable to a first external fluid, while being soluble in a second external fluid. This property can help to achieve a controlled and selective release of the active compound in the nucleus.

In some embodiments, the inert water soluble and/or erodible coat will be insoluble in the fluid of a first environment of use, such as gastric juices, acidic fluids, or polar liquids, and soluble or erodible in the fluid of a second environment of use, such as intestinal juices, substantially pH neutral or basic fluids, or apolar liquids. A wide variety of other polymeric materials are known to possess these various solubility properties and can be included in the water soluble and/or erodible coat. Such other polymeric materials include, by way of example and without limitation, cellulose acetate phthalate (CAP), cellulose acetate trimelletate (CAT), poly(vinyl acetate)phthalate (PVAP), hydroxypropyl methylcellulose phthalate (HP), poly(methacrylate ethylacrylate) (1:1) copolymer (MA-EA), poly(methacrylate methylmethacrylate) (1:1) copolymer (MA-MMA), poly(methacrylate methylmethacrylate) (1:2) copolymer, Eudragit™ L-30-D (MA-EA, 1:1), Eudragit™ L-100-55 (MA-EA, 1:1), hydroxypropylmethylcellulose acetate succinate (HPMCAS), Coateric™ (PVAP), Aquateric™ (CAP), AQOAT™ (HPMCAS) and combinations thereof. The coat can also comprise dissolution aids, stability modifiers, and bioabsorption enhancers.

An optional polymeric material for use in the inert water soluble and/or erodible coat includes enteric materials that resist the action of gastric fluid avoiding permeation through the semipermeable wall while one or more of the materials in the core are solubilized in the intestinal tract thereby allowing delivery of a drug in the core by osmotic pumping to begin. A material that easily adapts to this kind of requirement is a poly(vinylpyrrolidone)-vinyl acetate copolymer, such as the material supplied by BASF under its Kollidon VA64 trademark, mixed with magnesium stearate and other similar excipients. The water soluble and/or erodible coat can also comprise povidone, which is supplied by BASF under its Kollidon K 30 trademark, and hydroxypropyl methylcellulose, which is supplied by Dow under its Methocel E-15 trademark. The materials can be prepared in solutions having different concentrations of polymer according to the desired solution viscosity. For example, a 10% P/V aqueous solution of Kollidon™ K 30 has a viscosity of about 5.5-8.5 cps at 20.degree. C., and a 2% P/V aqueous solution of Methocel™ E-15 has a viscosity of about 13-18 cps at 20.degree. C.

The inert water soluble and/or erodible coat can also comprise other materials suitable which are substantially resistant to gastric juices and which will promote either enteric or colonic release. For this purpose, the inert water soluble and/or erodible coat can comprise one or more materials that do not dissolve, disintegrate, or change their structure in the stomach and during the period of time that the osmotic device resides in the stomach. Representative materials that keep their integrity in the stomach can comprise a member selected from the group consisting of (a) keratin, keratin sandarac-tolu, salol (phenyl salicylate), salol beta-naphthylbenzoate and acetotannin, salol with balsam of Peru, salol with tolu, salol with gum mastic, salol and stearic acid, and salol and shellac; (b) a member selected from the group consisting of formalized protein, formalized gelatin, and formalized cross-linked gelatin and exchange resins; (c) a member selected from the group consisting of myristic acid-hydrogenated castor oil-cholesterol, stearic acid-mutton tallow, stearic acid-balsam of tolu, and stearic acid-castor oil; (d) a member selected from the group consisting of shellac, ammoniated shellac, ammoniated shellac-salol, shellac-wool fat, shellac-acetyl alcohol, shellac-stearic acid-balsam of tolu, and shellac n-butyl stearate; (e) a member selected from the group consisting of abietic acid, methyl abictate, benzoin, balsam of tolu, sandarac, mastic with tolu, and mastic with tolu, and mastic with acetyl alcohol; (f) acrylic resins represented by anionic polymers synthesized from methacrylate acid and methacrylic acid methyl ester, copolymeric acrylic resins of methacrylic and methacrylic acid and methacrylic acid alkyl esters, copolymers of alkacrylic acid and alkacrylic acid alkyl esters, acrylic resins such as dimethylaminoethylmethacrylate-butylmethacrylate-methylmethacrylate copolymer of 150,000 molecular weight, methacrylic acid-methylmethacrylate 50:50 copolymer of 135,000 molecular weight, methacrylic acid-methylmethacrylate-30:70-copolymer of 135,000 mol. wt., methacrylic acid-dimethylaminoethyl-methacrylate-ethylacrylate of 750,000 mol. wt., methacrylic acid-methylmethacrylate-ethylacrylate of 1,000,000 mol. wt., and ethylacrylate-methylmethacrylate-ethylacrylate of 550,000 mol. wt; and, (g) an enteric composition comprising a member selected from the group consisting of cellulose acetyl phthalate, cellulose diacetyl phthalate, cellulose triacetyl phthalate, cellulose acetate phthalate, hydroxypropylmethylcellulose phathalate, sodium cellulose acetate phthalate, cellulose ester phthalate, cellulose ether phthalate, methylcellulose phthalate, cellulose ester-ether phthalate, hydroxypropyl cellulose phthalate, alkali salts of cellulose acetate phthalate, alkaline earth salts of cellulose acetate phthalate, calcium salt of cellulose acetate phthalate, ammonium salt of hydroxypropyl methylcellulose phthalate, cellulose acetate hexahydrophthalate, hydroxypropyl methylcellulose hexahydrophthalate, polyvinyl acetate phthalate diethyl phthalate, dibutyl phthalate, dialkyl phthalate wherein the alkyl comprises from 1 to 7 straight and branched alkyl groups, aryl phthalates, and other materials known to one or ordinary skill in the art.

The semipermeable membrane of the osmotic device is formed of a material that is substantially permeable to the passage of fluid from the environment of use to the core and substantially impermeable to the passage of active agent from the core. Many common materials that form a semipermeable wall which are known by those of ordinary skill in the art of pharmaceutical sciences are suitable for this purpose. Exemplary materials are cellulose esters, cellulose ethers and cellulose esters-ethers. However, it has been found that a semipermeable membrane comprising cellulose acetate (CA) and poly(ethylene glycol) (PEG), in particular PEG 400, performs well when used in combination with the other materials required in the present osmotic device. This particular combination of CA and PEG provides a semipermeable membrane that gives the osmotic device a well controlled release profile for the active agent in the core and that retains its chemical and physical integrity in the environment of use. The ratio of CA:PEG generally ranges from about 50-99% by weight of CA: about 50-1% by weight of PEG, and about 95% by weight of CA: about 5% by weight of PEG. The ratio can be varied to alter permeability and ultimately the release profile of the osmotic device. Other suitable materials can include a selected member of the group of cellulose acylates such as cellulose acetate, cellulose diacetate, cellulose triacetate and combinations thereof. Many suitable polymers, include those disclosed in Argentine Patent No. 199,301, U.S. Pat. No. 6,004,582 and references cited herein, the disclosures of which are hereby incorporated by reference.

Representative materials include a member selected from the group consisting of cellulose acylate, cellulose diacylate, cellulose triacylate, cellulose acetate, cellulose diacetate, cellulose triacetate, mono, di and tricellulose alkanylates, mono, di and tricellulose aroylates, and the like. Exemplary polymers include cellulose acetate having a D.S. up to 1 and an acetyl content up to 21%; cellulose acetate having an acetyl content of 32 to 39.8%; cellulose diacetate having a D.S. of 1 to 2 and an acetyl content of 21 to 35%; cellulose triacetate having a D.S. of 2 to 3 and an acetyl content of 35 to 44.8%; and the like. More specific cellulosic polymers include cellulose propionate having a D.S. of 1.8 and a propionyl content of 39.2 to 45% and a hydroxyl content of 2.8 to 5.4%; cellulose acetate butyrate having a D.S. of 1.8, an acetyl content of 13 to 15% and a butyryl content of 34 to 39%; cellulose acetate butyrate having an acetyl content of 2 to 29%; a butyryl content of 17 to 53% and a hydroxyl content of 0.5 to 4.7%; cellulose triacylates having a D.S. of 2.9 to 3 such as cellulose trivalerate, cellulose trilaurate, cellulose tripalmitate, cellulose trisuccinate, and cellulose trioclanoate; cellulose diacylates having a D.S. of 2.2 to 2.6 such as cellulose disuccinate, cellulose dipalmitate, cellulose dioclanoate, cellulose dipentale, and the like. Additional semipermeable polymers include acetaldehyde dimethyl acetate, cellulose acetate ethyl carbamate, cellulose acetate phthalate for use in environments having a low ph, cellulose acetate methyl carbamate, cellulose acetate dimethyl aminoacetate, semipermeable polyamides, semipermeable polyurethanes, semipermeable sulfonated polystyrenes, cross-linked selectively semipermeable polymers formed by the coprecipitation of a polyanion and a polycation as disclosed in U.S. Pat. No. 3,173,876, U.S. Pat. No. 3,276,586, U.S. Pat. No. 3,541,005, U.S. Pat. No. 3,541,006, and U.S. Pat. No. 3,546,142; semipermeable polymers as disclosed by Loeb and Sourirajan in U.S. Pat. No. 3,133,132; lightly cross-linked polystyrene derivatives; cross-linked poly(sodium styrene sulfonate), cross-linked poly(vinylbenzyltrimethyl ammonium chloride), semipermeable polymers exhibiting a fluid permeability of 10.sup.-5 to 10.sup.-1 (cc.mil/cm.sup.2.hr.atm) expressed as per atmosphere of hydrostatic or osmotic pressure difference across the semipermeable wall. These and others polymers are disclosed in U.S. Pat. No. 3,845,770, U.S. Pat. No. 3,916,899, U.S. Pat. No. 4,765,989 and U.S. Pat. No. 4,160,020; and in Handbook of Common Polymers (Scott, J. R. and Roff, W. J., eds.; 1971; CRC Press, Cleveland, Ohio).

The osmotic device of the invention comprises at least one passageway (pore, hole, or aperture) which communicates the exterior of the semipermeable wall with the core of the device. The passageway can be formed according to any of the known methods of forming passageways in a semipermeable membrane. Such methods include, for example, 1) drilling a hole through the semipermeable membrane with a bit or laser; 2) including a water soluble material within the composition that forms the semipermeable membrane such that a pore forms when the osmotic device is in an aqueous environment of use; 3) punching a hole through the semipermeable membrane; or 4) employing a tablet punch having a pin to punch a hole through the semipermeable lamina. The passageway can pass through the semipermeable wall and one or more of any other lamina coated onto the semipermeable membrane or between the semipermeable membrane and the core. The passageway(s) can be shaped as desired. In some embodiments, the passageway is laser drilled and is shaped as an oval, ellipse, slot, slit, cross or circle.

Methods of forming passageways in semipermeable membranes of osmotic devices are disclosed in U.S. Pat. No. 4,088,864 to Theeuwes et al., U.S. Pat. No. 4,016,880 to Theeuwes et al., U.S. Pat. No. 3,916,899 to Theeuwes et al., U.S. Pat. No. 4,285,987 to Ayer et al., U.S. Pat. No. 4,783,337 to Wong et al., U.S. Pat. No. 5,558,879 to Chen et al., U.S. Pat. No. 4,801,461 to Hamel et al., and U.S. Pat. No. 3,845,770 to Theeuwes et al., and U.S. Pregrant Patent Publication No. 2003-0189030, the disclosures of which are hereby incorporated by reference.

The core of the osmotic device tablet of the present invention will comprise licofelone, at least one pharmaceutically acceptable excipient and optionally one or more other materials. Generally, the tablet formulations will comprise about 0.1-99.9% by weight of licofelone in the uncoated tablet core. Acceptable ranges may vary according to the desired therapeutic response, the tablet size, the amount and type of excipients used in the core of the device, and the intended use of the osmotic device.

Osmotically effective solutes, osmotic agents or osmagents are added. These osmagents can aid in either the suspension or dissolution of the licofelone in the core. Exemplary osmagents include organic and inorganic compounds such as salts, acids, bases, chelating agents, sodium chloride, lithium chloride, magnesium chloride, magnesium sulfate, lithium sulfate, potassium chloride, sodium sulfite, calcium bicarbonate, sodium sulfate, calcium sulfate, calcium lactate, d-mannitol, urea, tartaric acid, raffinose, sucrose, alpha-d-lactose monohydrate, glucose, combinations thereof and other similar or equivalent materials which are widely known in the art. Osmagents can be incorporated to the core of the osmotic device to control the release of licofelone therefrom. U.S. Pat. No. 4,077,407 to Theeuwes et al., the entire disclosure of which is hereby incorporated by reference, discloses suitable osmagents.

The tablets of the invention can also comprise adsorbents, antioxidants, acidifying agent, alkalizing agent, buffering agents, colorants, flavorants, sweetening agents, tablet antiadherents, tablet binders, tablet diluents, tablet direct compression excipients, tablet disintegrants, tablet glidants, tablet lubricants, tablet opaquants and/or tablet polishing agents.

As used herein, the term “alkalizing agent” is intended to mean a compound used to provide alkaline medium for product stability. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, diethanolamine, organic amine base, alkaline amino acids and trolamine and others known to those of ordinary skill in the art.

As used herein, the term “acidifying agent” is intended to mean a compound used to provide an acidic medium for product stability. Such compounds include, by way of example and without limitation, acetic acid, acidic amino acids, citric acid, fumaric acid and other alpha hydroxy acids, hydrochloric acid, ascorbic acid, phosphoric acid, sulfuric acid, tartaric acid and nitric acid and others known to those of ordinary skill in the art.

As used herein, the term “adsorbent” is intended to mean an agent capable of holding other molecules onto its surface by physical or chemical (chemisorption) means. Such compounds include, by way of example and without limitation, powdered and activated charcoal and other materials known to one of ordinary skill in the art.

As used herein, the term “antioxidant” is intended to mean an agent which inhibits oxidation and thus is used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate and sodium metabisulfite and other materials known to one of ordinary skill in the art.

As used herein, the term “buffering agent” is intended to mean a compound used to resist change in pH upon dilution or addition of acid or alkali. Such compounds include, by way of example and without limitation, potassium metaphosphate, potassium phosphate, monobasic sodium acetate and sodium citrate anhydrous and dihydrate and other materials known to one of ordinary skill in the art.

As used herein, the term “sweetening agent” is intended to mean a compound used to impart sweetness to a preparation. Such compounds include, by way of example and without limitation, aspartame, dextrose, glycerin, mannitol, saccharin sodium, sorbitol and sucrose and other materials known to one of ordinary skill in the art.

As used herein, the term “antiadherents” is intended to mean agents which prevent the sticking of tablet formulation ingredients to punches and dies in a tableting machine during production. Such compounds include, by way of example and without limitation, magnesium stearate, talc, calcium stearate, glyceryl behenate, PEG, hydrogenated vegetable oil, mineral oil, stearic acid and other materials known to one of ordinary skill in the art.

As used herein, the term “binders” is intended to mean substances used to cause adhesion of powder particles in table granulations. Such compounds include, by way of example and without limitation, acacia, alginic acid, carboxymethylcellulose sodium, poly(vinylpyrrolidone), compressible sugar (e.g., NuTab), ethylcellulose, gelatin, liquid glucose, methylcellulose, povidone and pregelatinized starch and other materials known to one of ordinary skill in the art.

When needed, binders may also be included in the tablets. Exemplary binders include acacia, tragacanth, gelatin, starch, cellulose materials such as methyl cellulose and sodium carboxy methyl cellulose, alginic acids and salts thereof, polyethylene glycol, guar gum, polysaccharide, bentonites, sugars, invert sugars, poloxamers (PLURONIC™ F68, PLURONIC™ F127), collagen, albumin, gelatin, cellulosics in nonaqueous solvents, combinations thereof and the like. Other binders include, for example, polypropylene glycol, polyoxyethylene-polypropylene copolymer, polyethylene ester, polyethylene sorbitan ester, polyethylene oxide, combinations thereof and other materials known to one of ordinary skill in the art.

As used herein, the term “diluent” or “filler” is intended to mean inert substances used as fillers to create the desired bulk, flow properties, and compression characteristics in the preparation of tablets and capsules. Such compounds include, by way of example and without limitation, dibasic calcium phosphate, kaolin, lactose, sucrose, mannitol, microcrystalline cellulose, powdered cellulose, precipitated calcium carbonate, sorbitol, and starch and other materials known to one of ordinary skill in the art.

As used herein, the term “direct compression excipient” is intended to mean a compound used in direct compression tablet formulations. Such compounds include, by way of example and without limitation, dibasic calcium phosphate (e.g., Ditab) and other materials known to one of ordinary skill in the art.

As used herein, the term “glidant” is intended to mean agents used in tablet and capsule formulations to promote flowability of the granulation. Such compounds include, by way of example and without limitation, colloidal silica, cornstarch, talc, calcium silicate, magnesium silicate, colloidal silicon, silicon hydrogel and other materials known to one of ordinary skill in the art.

As used herein, the term “lubricant” is intended to mean substances used in tablet formulations to reduce friction during tablet compression. Such compounds include, by way of example and without limitation, calcium stearate, magnesium stearate, mineral oil, stearic acid, and zinc stearate and other materials known to one of ordinary skill in the art.

As used herein, the term “opaquant” is intended to mean a compound used to render a capsule or a tablet coating opaque. May be used alone or in combination with a colorant. Such compounds include, by way of example and without limitation, titanium dioxide, talc and other materials known to one of ordinary skill in the art.

As used herein, the term “polishing agent” is intended to mean a compound used to impart an attractive sheen to coated tablets. Such compounds include, by way of example and without limitation, camauba wax, white wax and other materials known to one of ordinary skill in the art.

As used herein, the term “disintegrant” is intended to mean a compound used in solid dosage forms to promote the disruption of the solid mass into smaller particles which are more readily dispersed or dissolved. Exemplary disintegrants include, by way of example and without limitation, starches such as corn starch, potato starch, pre-gelatinized and modified starches thereof, sweeteners, clays, such as bentonite, microcrystalline cellulose(e.g., Avicel), carboxymethylcellulose calcium, cellulose polyacrilin potassium (e.g., Amberlite), alginates, sodium starch glycolate, gums such as agar, guar, locust bean, karaya, pectin, tragacanth; crospovidone and other materials known to one of ordinary skill in the art.

As used herein, the term “colorant” is intended to mean a compound used to impart color to solid (e.g., tablets) pharmaceutical preparations. Such compounds include, by way of example and without limitation, FD&C Red No. 3, FD&C Red No. 20, FD&C Yellow No. 6, FD&C Blue No. 2, D&C Green No. 5, D&C Orange No. 5, D&C Red No. 8, caramel, and ferric oxide, red, other F.D. & C. dyes and natural coloring agents such as grape skin extract, beet red powder, beta-carotene, annato, carmine, turmeric, paprika, and other materials known to one of ordinary skill in the art. The amount of coloring agent used will vary as desired.

As used herein, the term “flavorant” is intended to mean a compound used to impart a pleasant flavor and often odor to a pharmaceutical preparation. Exemplary flavoring agents or flavorants include synthetic flavor oils and flavoring aromatics and/or natural oils, extracts from plants, leaves, flowers, fruits and so forth and combinations thereof. These may also include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. Other useful flavors include vanilla, citrus oil, including lemon, orange, grape, lime and grapefruit, and fruit essences, including apple, pear, peach, strawberry, raspberry, cherry, plum, pineapple, apricot and so forth. Flavors which have been found to be particularly useful include commercially available orange, grape, cherry and bubble gum flavors and mixtures thereof. The amount of flavoring may depend on a number of factors, including the organoleptic effect desired. Flavors will be present in any amount as desired by those of ordinary skill in the art. Particularly flavors are the grape and cherry flavors and citrus flavors such as orange.

The present tablets can also employ one or more commonly known surface active agents or cosolvents that improve wetting or disintegration of the tablet core or layers.

Plasticizers can also be included in the tablets to modify the properties and characteristics of the polymers used in the coats or core of the tablets. As used herein, the term “plasticizer” includes all compounds capable of plasticizing or softening a polymer or binder used in invention. The plasticizer should be able to lower the melting temperature or glass transition temperature (softening point temperature) of the polymer or binder. Plasticizers, such as low molecular weight PEG, generally broaden the average molecular weight of a polymer in which they are included thereby lowering its glass transition temperature or softening point. Plasticizers also generally reduce the viscosity of a polymer. It is possible the plasticizer will impart some particularly advantageous physical properties to the osmotic device of the invention.

Plasticizers useful in the invention can include, by way of example and without limitation, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene glycol), citrate ester-type plasticizers, triacetin, propylene glycol and glycerin. Such plasticizers can also include ethylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutylsebacate, acetyltributylcitrate, triethyl citrate, acetyl triethyl citrate, tributyl citrate and allyl glycolate. All such plasticizers are commercially available from sources such as Aldrich or Sigma Chemical Co. It is also contemplated and within the scope of the invention, that a combination of plasticizers may be used in the present formulation. The PEG based plasticizers are available commercially or can be made by a variety of methods, such as disclosed in Poly(ethylene glycol) Chemistry: Biotechnical and Biomedical Applications (J. M. Harris, Ed.; Plenum Press, NY) the disclosure of which is hereby incorporated by reference.

The tablets of the invention can also include oils, for example, fixed oils, such as peanut oil, sesame oil, cottonseed oil, corn oil and olive oil; fatty acids, such as oleic acid, stearic acid and isotearic acid; and fatty acid esters, such as ethyl oleate, isopropyl myristate, fatty acid glycerides and acetylated fatty acid glycerides. It can also be mixed with alcohols, such as ethanol, isopropanol, hexadecyl alcohol, glycerol and propylene glycol; with glycerol ketals, such as 2,2-dimethyl-1,3-dioxolane-4-methanol; with ethers, such as poly(ethyleneglycol) 450, with petroleum hydrocarbons, such as mineral oil and petrolatum; with water, or with mixtures thereof; with or without the addition of a pharmaceutically suitable surfactant, suspending agent or emulsifying agent.

Soaps and synthetic detergents may be employed as surfactants and as vehicles for detergent compositions. Suitable soaps include fatty acid alkali metal, ammonium, and triethanolamine salts. Suitable detergents include cationic detergents, for example, dimethyl dialkyl ammonium halides, alkyl pyridinium halides, and alkylamine acetates; anionic detergents, for example, alkyl, aryl and olefin sulfonates, alkyl, olefin, ether and monoglyceride sulfates, and sulfosuccinates; nonionic detergents, for example, fatty amine oxides, fatty acid alkanolamides, and poly(oxyethylene)-block-poly(oxypropylene) copolymers; and amphoteric detergents, for example, alkyl β-aminopropionates and 2-alkylimidazoline quaternary ammonium salts; and mixtures thereof.

Various other components, not otherwise listed above, can be added to the present formulation for optimization of a desired active agent release profile including, by way of example and without limitation, glycerylmonostearate, nylon, cellulose acetate butyrate, d, 1-poly(lactic acid), 1,6-hexanediamine, diethylenetriamine, starches, derivatized starches, acetylated monoglycerides, gelatin coacervates, poly (styrene-maleic acid) copolymer, glycowax, castor wax, stearyl alcohol, glycerol palmitostearate, poly(ethylene), poly(vinyl acetate), poly(vinyl chloride), 1,3-butylene-glycoldimethacrylate, ethyleneglycol-dimethacrylate and methacrylate hydrogels.

It should be understood, that compounds used in the art of pharmaceutical formulation generally serve a variety of finctions or purposes. Thus, if a compound named herein is mentioned only once or is used to define more than one term herein, its purpose or function should not be construed as being limited solely to that named purpose(s) or function(s).

The tablets of the invention can assume any shape or form known in the art of pharmaceutical sciences. The device of the invention can be a pill, sphere, tablet, bar, plate, paraboloid of revolution, ellipsoid of revolution or the like. The tablets can also include surface markings, cuttings, grooves, letters and/or numerals for the purposes of decoration, identification and/or other purposes.

The tablets of the invention can be prepared according to the methods disclosed herein or those well known in the art, more specifically according to the methods disclosed in the disclosure incorporated herein by reference. For example, according to one manufacturing technique, licofelone and excipients that comprise the core are mixed in solid, semisolid or gelatinous form, then moistened and sieved through a specified screen to obtain granules. The granules are then dried in a dryer and compressed, for example, by punching to form uncoated cores. The compressed and uncoated cores are then covered with a semipermeable membrane. Subsequently, the semipermeable membrane surrounding the core should be perforated with, for example, laser equipment. Optionally, an external coat containing licofelone is applied to the semipermeable membrane.

The external coat can be applied as a compression coating, but it is generally applied as a sprayed coating. The sprayed coating is thinner and lighter than the compression coating, and an osmotic device including the sprayed on external coating is, therefore, smaller than a similar osmotic device having a compression coat. Moreover, the use of a sprayed-on drug-containing water soluble coating permits the loading of higher amounts of drug than the use of a compression-coated drug-containing water soluble coating. A smaller size osmotic device generally results in increased patient compliance in taking the osmotic device and is therefore advantageous.

The tablets of the invention can be coated with a finish coat as is commonly done in the art to provide the desired shine, color, taste or other aesthetic characteristics. Materials suitable for preparing the finish coat are well known in the art and found in the disclosures of many of the references cited and incorporated by reference herein.

The osmotic device of the invention is used to treat a disease or disorder that is responsive to treatment with licofelone. Exemplary disorders include osteoarthritis, rheumatoid arthritis, and an inflammation related disorder.

The osmotic device of the invention would be useful for, but not limited to, the treatment of arthritis, rheumatoid arthritis, spondyloarthopathies, gouty arthritis, osteoarthritis, systemic lupus erythematosus and juvenile arthritis. The invention would also be useful in the treatment of asthma, bronchitis, menstrual cramps, tendinitis, bursitis, and skin related conditions such as psoriasis, eczema, burns and dermatitis. The invention also would be useful to treat gastrointestinal conditions such as inflammatory bowel disease, Crohn's disease, gastritis, irritable bowel syndrome and ulcerative colitis and for the prevention or treatment of cancer, such as colorectal cancer. And, the invention would be useful in treating inflammation in such diseases as vascular diseases, migraine headaches, periarteritis nodosa, thyroiditis, aplastic anemia, Hodgkin's disease, sclerodoma, rheumatic fever, type I diabetes, myasthenia gravis, multiple sclerosis, sarcoidosis, nephrotic syndrome, Behcet's syndrome, polymyositis, gingivitis, hypersensitivity, swelling occurring after injury, myocardial ischemia, viral infections and cystic fibrosis; central nervous system disorders such as cortical dementias including Alzheimer's disease; ; allergic diseases, allergic rhinitis, respiratory distress syndrome, endotoxin shock syndrome, atherosclerosis and central nervous system damage resulting from stroke, ischemia and trauma.

The invention thus provides a method of treating the disease or disorder in a subject by orally administering to the subject an osmotic device as described herein. One or two osmotic device adapted for once daily administration can be administered. Generally, a single osmotic device is administered once daily; however, two osmotic device can be administered to a subject within a 24-hour period if the dose in a single osmotic device is too low for that subject. The osmotic device is adapted to release drug in a controlled manner for a period drug for a period of at least about 18 hours. If two osmotic devices of the invention are administered to a subject, they can be administered substantially at the same time or in a sequential manner, wherein the second osmotic device is administered about one hour after administration of the first osmotic device.

Practice of the method of the invention results in an improved clinical benefit over administration of licofelone in immediate (rapid) release form. Improvements include reduced occurrence and/or severity of side-effects associated with licofelone therapy, reduced plasma peak-trough fluctuation, steady-state plasma licofelone concentrations sufficient to provide effective therapy when administered at dosing intervals of about 24 hours, half or less than half of the number of peak plasma licofelone concentrations each 24-hour period compared to the number that occurs with twice-a-day administration of an immediate-release licofelone dosage form, and convenience to the patient if once daily administration is available versus twice a day dosing.

The following examples should not be considered exhaustive, but merely illustrative of only a few of the many embodiments contemplated by the present invention. The methods described herein can be followed to prepare osmotic devices according to the invention.

### EXAMPLE 1

The following procedure is used to prepare an osmotic device tablets containing Licofelone (200, 400, and 800 mg strength) in the osmotic device. The osmotic device tablets contain the following ingredients in the amounts indicated:

| Ingredients | Amount (mg) | | |
|---|---|---|---|
| Licofelone Strength [symbol] | 200.000 | 400.00 | 800.00 |
| CORE | | | |
| Licofelone | 200.00 | 400.00 | 800.00 |
| Sodium Chloride | 50.00 | 100.00 | 200.00 |
| Polyethylene Oxide 205 NF | 30.00 | 60.00 | 120.00 |
| Povidone | 2.00 | 4.00 | 8.00 |
| Hydroxypropyl methylcellulose 2208 | 2.00 | 4.00 | 8.00 |
| Polyethylene Glycol 400 | 1.50 | 3.00 | 6.00 |
| Cellulose Microcrystalline | 12.00 | 24.00 | 48.00 |
| Colloidal Silicon Dioxide | 1.00 | 2.00 | 4.00 |
| Magnesium Stearate | 1.50 | 3.00 | 6.00 |
| Purified water | 20.00 | 40.00 | 60.00 |
| COATING A | | | |
| Cellulose Acetate 398 | 19.00 | 23.75 | 28.50 |
| Polyethylene Glycol 400 | 1.00 | 1.25 | 1.50 |
| Acetone | 400.00 | 500.00 | 600.00 |
| COATING B | | | |
| Opadry 1 | 10.00 | 15.00 | 20.00 |
| Purified Water | 130.00 | 195.00 | 260.00 |

First, the core composition is prepared by placing licofelone, sodium chloride, microcrystalline cellulose, hydroxypropyl methylcellulose 2208 (Methocel K 4M), polyethylene oxide 205 NF, and povidone in a high shear mixer and mix for 5 minutes. The granulation process is initiated by the gradual addition of a granulating solution containing polyethylene glycol 400 and purified water to the high shear with continuous blending to produce a wet blend. Next, the wet blend is granulated and dried at 40-50° C. for 20 minutes in a fluid bed to remove the water. Then, the dry granules are screened through a 30 USP mesh screen for size reduction. Next, the screened granules are mixed with the colloidal silicon dioxide and magnesium stearate, that have been previously passed through a 60 mesh screen, in a V-blender during 5 minutes. This final blend is tabletted to provide the cores.

A first composition to cover the coated cores is prepared as follows: cellulose acetate 398 and polyethylene glycol 400 are added to acetone and mixed thoroughly to form a polymer solution. This solution is sprayed onto the tablets in a perforated pan coater to form semipermeable membrane coated cores. A 0.5 mm hole is drilled through the coating to provide perforated cores.

A finish coat comprising Opadry in purified water is applied onto the film-coated tablets to obtain the multi-layered osmotic device tablets.

### EXAMPLE 2

The following procedure is used to prepare multi-layered osmotic device tablets containing licofelone (150, 300, and 600 mg strength) in the osmotic core and licofelone (50, 100, and 200 mg strength, respectively) in a drug-containing external coat of the osmotic device. The osmotic device tablets contain the following ingredients in the amounts indicated:

| Ingredients | Amount (mg) | | |
|---|---|---|---|
| Licofelone Strength [symbol] | 200.00 | 400.00 | 800.00 |
| CORE | | | |
| Licofelone | 150.00 | 300.00 | 600.00 |
| Sodium Chloride | 37.50 | 75.00 | 150.00 |
| Polyethylene Oxide 205 NF | 22.50 | 45.00 | 90.00 |
| Povidone | 1.50 | 3.00 | 6.00 |
| Hydroxypropyl methylcellulose 2208 | 1.50 | 3.00 | 6.00 |
| Polyethylene Glycol 400 | 1.13 | 2.26 | 4.52 |
| Cellulose Microcrystalline | 9.00 | 18.00 | 36.00 |
| Colloidal Silicon Dioxide | 0.75 | 1.50 | 3.00 |
| Magnesium Stearate | 1.13 | 2.26 | 4.52 |
| Purified water | 15.00 | 30.00 | 60.00 |
| COATING A | | | |
| Cellulose Acetate 398 | 16.60 | 21.38 | 28.50 |
| Polyethylene Glycol 400 | 0.87 | 1.13 | 1.50 |
| Acetone | 349.47 | 450.11 | 600.00 |
| COATING B | | | |
| Titanium Dioxide | 1.50 | 2.50 | 4.00 |
| Talc | 3.75 | 6.25 | 10.00 |
| Povidone | 2.25 | 3.75 | 6.00 |
| Purified water | 22.50 | 37.50 | 60.00 |
| COATING C | | | |
| Licofelone | 50.00 | 100.00 | 200.00 |
| HPMC 2910 | 12.50 | 25.00 | 50.00 |
| Crospovidone | 10.50 | 21.00 | 42.00 |
| Polyethylene Glycol 6000 | 3.50 | 7.00 | 14.00 |
| Colloidal Silicon Dioxide | 1.00 | 2.00 | 4.00 |
| Purified Water | 135.00 | 270.00 | 540.00 |
| COATING D | | | |
| Opadry 1 | 7.20 | 12.00 | 19.20 |
| Purified Water | 93.60 | 156.00 | 249.60 |

Licofelone, sodium chloride, microcrystalline cellulose, hydroxypropyl methylcellulose 2208 (Methocel K 4M), polyethylene oxide 205 NF and povidone are mixed in a high shear mixer for 5 minutes. The granulation process is initiated by the gradual addition of a granulating solution containing polyethylene glycol 400 and purified water to the high shear with continuous blending to produce a wet blend. Next, the wet blend is granulated and dried at 40-50° C. for 20 minutes in a fluid bed to remove the water. Then, the dry granules are screened through a 30 USP mesh screen for size reduction. Next, the screened granules are mixed with the colloidal silicon dioxide and magnesium stearate, that have been previously passed through a 60 mesh screen, in a V-blender during 5 minutes. This final blend is tabletted to provide the cores.

A first composition to cover the coated cores is prepared as follows: cellulose acetate 398 and polyethylene glycol 400 are added to acetone and mixed thoroughly to form a polymer solution. This solution is sprayed onto the tablets in a perforated pan coater to form semipermeable membrane coated cores. A 0.5 mm hole is drilled through the coating to provide perforated cores.

A second composition to cover the perforated cores is prepared as follows: povidone, titanium dioxide and talc are added to the purified water to form the coating suspension. This suspension is sprayed onto the tablets in a perforated pan coater to obtain drug load coated tablets.

A third composition to cover the perforated cores is prepared as follows: licofelone, HPMC 2910, crospovidone, colloidal silicon dioxide and polyethylene glycol 6000 are added to the purified water to form the coating suspension. This suspension is sprayed onto the tablets in a perforated pan coater to obtain drug load coated tablets.

A finish coat comprising Opadry in purified water is applied onto the film-coated tablets to obtain the multi-layered osmotic device tablets.

### EXAMPLE 3

The pharmacokinetics of licofelone dosage forms in accord with the present invention and conventional immediate release dosage forms were compared in a randomized, open-label, single dose, two-way crossover study in 12 healthy male and female subjects. The reference treatment consisted of a single 200 mg dose of licofelone in an immediate release dosage form. The test treatment consisted of a single dose of 400 mg of licofelone osmotic device of Example 1. For the purposes of this disclosure, the following definitions shall apply:

Cmax: Peak drug concentration, obtained directly from the plasma concentration-time curve.

Tmax: The time to attain the peak drug concentration, which was obtained directly form the plasma concentration-time curve.

λ_{z}: The terminal or the elimination rate constant was calculated according to the linear regression analysis of log-concentration versus time.

T1/2: The terminal or the elimination half-life of the drug was calculated as ln(2)/λ_{z}.

AUClast: Area under the curve from zero to the last non-zero measurable concentration calculated through the linear trapezoidal rule. This value was chosen as the representative experimental AUC.

AUCinf: Area under the plasma concentration-time curve extrapolated to infinity as AUClast+Clast/λ_{z}.

Relative Bioavailability (%): A measure of total exposure to the drug in regards to a reference product. Calculated as

$$\% R = 100 \frac{{AUC}_{inf}^{CR} \times {D}^{IR}}{{AUC}_{inf}^{IR} \times {D}^{CR}}$$

Cmax,ss: Peak drug concentration, obtained directly from the plasma concentration-time curve.

Cmin,ss: Though drug concentration, obtained directly from the plasma concentration-time curve.

Tau: period between 2 consecutive doses Cav,ss: Average drug concentration at steady state, calculated as AUCinf/Tau.

Fluctutation(%): Calculated as 100*(Cmax,ss−Cmin,ss) / Cav,ss

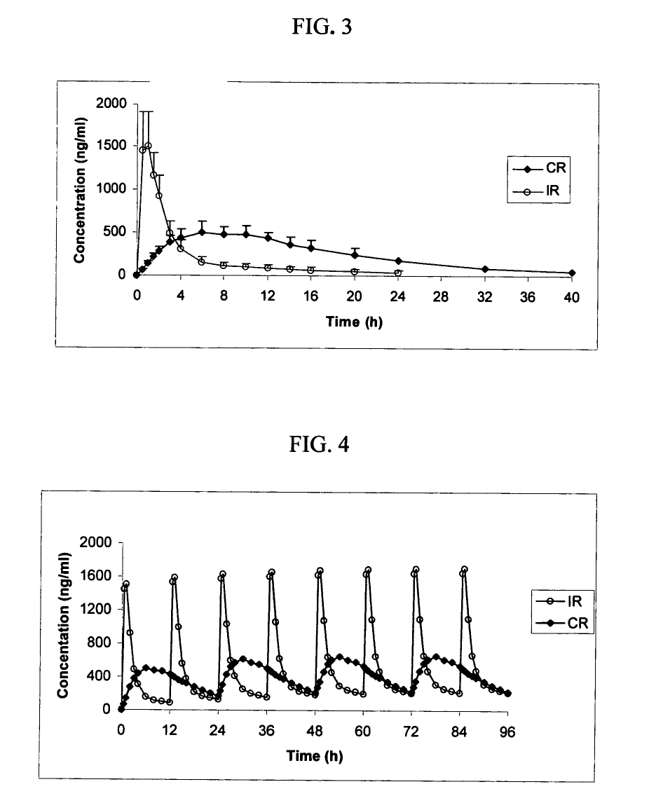

Each single-dose treatment was followed by a seven-day washout period. The resulting plasma licofelone concentration profiles are shown in FIG. 3 (open circles for the immediate release (IR) dosage form, closed diamonds for the sustained release (CR) dosage form). The mean Cmax (ng/ml) values were as follows: 1568.8 for the immediate release dosage form and 520.8 for the sustained release dosage form. The mean Tₘₐₓ (h) following administration of the immediate-release dosage form was just 1.4 hours while following administration of the sustained release dosage forms the mean Tₘₐₓ values were 7.3 hours.

Bioavailability of the sustained release formulation relative to the immediate release dosage form was 90.9%.

The main pharmacokinetic parameters (mean±S.D., n=12) are shown in Table 1 below:

TABLE 1

| Parameter | Units | CR (400 mg) | IR (200 mg) |
|---|---|---|---|
| T½ | H | 10.1 ± 1.8 | 11.1 ± 2.5 |
| Tmax | H | 7.3 ± 2.0 | 1.4 ± 0.5 |
| Cmax | ng/ml | 520.8 ± 116.0 | 1568.8 ± 433.7 |
| AUCinf | ng*hr/ml | 10704.7 ± 2913.1 | 5963.8 ± 1835.2 |

Mean steady-state plasma licofelone concentrations, following dosing every 24 hours with 400 mg doses of licofelone osmotic device of Example 1, and following dosing every 12 hours with 200 mg doses of licofelone (total daily dose=400 mg) in an immediate release dosage form for a period of four days (96 hours) were simulated. The simulated plasma licofelone concentration profiles are shown in graph form in FIG. 4 (open circles for the immediate release dosage form, closed diamonds for the sustained release dosage form). It can be seen that peak plasma licofelone concentrations are lower following administration of the sustained release dosage form than those observed following administration of the immediate release dosage form. Additionally, the number of peak plasma licofelone concentrations occurring over the four day period with the sustained release dosage form regimens are half the number occurring with the immediate release dosage form regimen, i.e., 4 vs 8.

Fluctuation of steady-state licofelone plasma concentrations was estimated to be 275% after a regimen of administration of 200 mg of licofelone as an immediate release dosage form every 12 hours. Fluctuation was estimated to be 94% after a regimen of administration of 400 mg of licofelone as a sustained release form.

The mean licofelone plasma concentrations in ng/ml following two different regimen of administration are shown in Table 2 below:

TABLE 2

| Dose Regimen | Cmax | Cmin | Cav |
|---|---|---|---|
| 200 mg immediate release | 1705 | 223 | 539 |
| administered every 12 hours | | | |
| 400 mg sustained release | 650 | 223 | 452 |
| administered every 24 hours | | | |

In the simulation, the time to steady-state peak plasma licofelone concentrations is significantly different for the sustained release licofelone dosage form compared to the immediate release dosage form. The immediate release dosage form peaks only 0.8 hours following dose administration while the sustained release dosage form exhibits a delayed peak of 6 hours. As a result, therapeutic plasma levels are obtained from the sustained release dosage form beyond 6 hours after dosing, which are approximately twice the concentrations produced by the immediate release dosage form. Additionally, the lower peak concentration of the controlled release dosage forms would reduce the occurrence and/or severity of side effects. Accordingly, the dosage form of the invention provides therapeutically effective plasma concentrations of licofelone for a longer period than does a rapid release dosage form containing the same amount of licofelone. The dosage form of the invention provides therapeutically effective plasma concentrations of licofelone for a period of at least 6, 8, 10, 12, 14, 16, 18, 20, 22 or 24 hours.

### EXAMPLE 4

A multi-layered osmotic device tablet containing licofelone (150-600 mg strength) in the osmotic core and licofelone (50-200 mg strength) in a drug-containing external coat of the osmotic device can be prepared as described herein. The following ingredients in the amounts indicated are used to make a multi-layered osmotic device tablet of the invention.

| Ingredients | Amount (mg) |
|---|---|
| Total Licofelone Strength [symbol] | 200-800 |
| CORE | |
| Licofelone | 150-600 |
| Osmagent | 35-150 |
| Osmopolymer | 20-90 |
| Binder | 1-6 |
| Water soluble polymer | 1-6 |
| Plasticizer | 1-5 |
| Disintegrant | 9-36 |
| Glidant | 0.5-3 |
| Lubricant | 1-5 |
| Purified water | 10-60 |
| COATING A (semipermeable coat) | |
| Semipermeable film-forming polymer | 15-30 |
| Plasticizer | 0.5-2.0 |
| Solvent | 300-600 |
| COATING B (inert coat) | |
| Opaquant | 1-5 |
| Water erodible material | 3-10 |
| Water soluble polymer | 2-6 |
| Purified water | 20-60 |
| COATING C (optional, drug-containing coat) | |
| Licofelone | 50-200 |
| Water soluble polymer | 10-50 |
| Water erodible polymer | 10-45 |
| Plasticizer | 3-15 |
| Glidant | 1-5 |
| Purified Water | 135-550 |
| COATING D (optional, finish coat) | |
| Water soluble polymer | 7-20 |
| Purified Water | 90-250 |

The following procedure or another similar procedure in the art of osmotic devices can be used to prepare an osmotic device with the above-noted ingredients. It should be noted that the water and solvents listed in the above table are present during initial manufacture of each element of the osmotic device; however, all or almost all of the water and solvents are absent in the fmal dosage form. In other words, the final form of the osmotic device of the invention generally comprises less than 10% wt. or less than 5% wt. of water or solvent, the water and solvent having been removed during manufacture of the osmotic device.

Licofelone, an osmagent, a desintegrant, a water soluble polymer, an osmopolymer and a binder are mixed in a high shear mixer for 5 minutes. The granulation process is initiated by the gradual addition of a granulating solution containing a plasticizer and purified water to the high shear with continuous blending to produce a wet blend. Next, the wet blend is granulated and dried at 40-50° C. for 20 minutes in a fluid bed to remove the water. Then, the dry granules are screened through a 30 USP mesh screen for size reduction. Next, the screened granules are mixed with a glidant and a lubricant, that have been previously passed through a 60 mesh screen, in a V-blender during 5 minutes. This final blend is tabletted to provide the cores.

A first composition to cover the coated cores is prepared as follows: a semipermeable film-forming polymer and a plasticizer are added to an organic solvent and mixed thoroughly to form a polymer solution. This solution is sprayed onto the tablets in a perforated pan coater to form semipermeable membrane coated cores. A 0.5 mm hole is drilled through the coating to provide perforated cores.

A second composition to cover the perforated cores is prepared as follows: an opaquant, a water erodible material and a water soluble polymer are added to the purified water to form the coating suspension. This suspension is sprayed onto the tablets in a perforated pan coater to obtain drug load coated tablets.

A third composition to cover the perforated cores is prepared as follows: licofelone, a water soluble polymer, a water erodible polymer, a plasticizer, and a glidant are added to the purified water to form the coating suspension. This suspension is sprayed onto the tablets in a perforated pan coater to obtain drug load coated tablets.

A finish coat comprising a water soluble polymer in purified water is applied onto the film-coated tablets to obtain the multi-layered osmotic device tablets.

The above is a detailed description of particular embodiments of the invention. It is recognized that departures from the disclosed embodiments may be made within the scope of the invention and that obvious modifications will occur to a person skilled in the art. Those of skill in the art should, in light of the present disclosure, appreciate that many changes can be made in the specific embodiments which are disclosed herein and still obtain a like or similar result without departing from the spirit and scope of the invention. All of the embodiments disclosed and claimed herein can be made and executed without undue experimentation in light of the present disclosure.

1. An osmotic device comprising:
a core comprising a first amount licofelone and at least one osmotic agent or osmopolymer;
a semipermeable membrane surrounding the core and having at least one passageway there through; and
optionally, a drug-containing water soluble and/or erodible coat comprising a second amount of licofelone;
wherein at least 75% of the licofelone is released within 24 hours after exposure of the osmotic device to an aqueous environment, and wherein the first and second amounts together comprise a therapeutically effective amount suitable for once daily administration to a subject.

2. The osmotic device of claim 1, wherein: 1) at least 10% of the licofelone is released from the core within 4 hours; 2) at least 45% of the licofelone is released from the core within 12 hours; 3) at least 60% of the licofelone is released from the core within 16 hours; and 4) at least 75% of the licofelone is released from the core within 20 hours after exposure of the osmotic device to an aqueous environment.

3. The osmotic device of claim 1, wherein: 1) 20-30% of the licofelone is released within about 1 hours; 2) 25-65% of the licofelone is released within about 4 hours; 3) 47-83% of the licofelone is released within about 12 hours; and 4) at least 75% of the licofelone is released within about 24 hours after exposure of the osmotic device to an aqueous environment.

4. The osmotic device of any one of claims 1, 2 or 3, wherein the drug-containing coat is present, further comprising an inert water soluble and/or erodible coating disposed between the semipermeable membrane and the drug-containing coating.

5. The osmotic device of claim 4, wherein the drug-containing coat is sprayed onto the inert coating.

6. The osmotic device of claims 1 or 2 wherein the licofelone is released from the core according to a first order or pseudo-first order rate for a period of at least 12 hours.

7. The osmotic device of claim 4 wherein the licofelone is released from the core according to a first order or pseudo-first order rate for a period of at least 12 hours.

8. The osmotic device of claims 1 or 2 wherein the licofelone is released from the core according to a zero order or pseudo-zero order rate for a period of at least 12 hours.

9. The osmotic device of claim 4 wherein the licofelone is released from the core according to a zero order or pseudo-zero order rate for a period of at least 12 hours.

10. The osmotic device of claims 1 or 2 wherein the licofelone is released from the core according to a sigmoidal release profile.

11. The osmotic device of claims 4 wherein the licofelone is released from the core according to a sigmoidal release profile.

12. The osmotic device of claim 4 wherein the drug-containing coat is present in an amount of at least about 25% wt. based upon the total weight of the osmotic device.

13. A method of treating a condition in a subject, the condition being responsive to treatment with licofelone, the method comprising the step of administering to the subject once per day an osmotic device comprising:
a core comprising a first amount licofelone and at least one osmotic agent or osmopolymer;
a semipermeable membrane surrounding the core and having at least one passage way there through; and
optionally, a drug-containing water soluble and/or erodible coat comprising a second amount of licofelone;
wherein at least 75% of licofelone is released within 24 hours after exposure of the osmotic device to an aqueous environment and wherein the first and second amounts together comprise a therapeuticay effective amount suitable for once daily administration to a subject.

14. The method of claim 13, wherein the condition is selected from the group consisting of osteoarthritis, rheumatoid arthritis, an inflammation related disorder, spondyloarthopathies, gouty arthritis, osteoarthritis, systemic lupus erythematosus and juvenile arthritis, asthma, bronchitis, menstrual cramps, tendinitis, bursitis; skin related conditions such as psoriasis, eczema, burns and dermatitis; gastrointestinal conditions such as inflammatory bowel disease, Crohn's disease, gastritis, irritable bowel syndrome and ulcerative colitis; for the prevention or treatment of cancer, such as colorectal cancer; inflammation in vascular disease, migraine headache, periarteritis nodosa, thyroiditis, aplastic anemia, Hodgkin's disease, sclerodoma, rheumatic fever, type I diabetes, myasthenia gravis, multiple sclerosis, sarcoidosis, nephrotic syndrome, Behcet's syndrome, polymyositis, gingivitis, hypersensitivity, swelling occurring after injury, myocardial ischemia, viral infections and cystic fibrosis; central nervous system disorders such as cortical dementias including Alzheimer's disease; allergic diseases; allergic rhinitis; respiratory distress syndrome; endotoxin shock syndrome; atherosclerosis; and central nervous system damage resulting from stroke, ischemia and trauma.

15. The method of claim 14, wherein: 1) at least 10% of the licofelone is released from the core within 4 hours; 2) at least 45% of the licofelone is released from the core within 12 hours; 3) at least 60% of the licofelone is released from the core within 16 hours; and at least 75% of the licofelone is released from the core within 20 hours after exposure of the osmotic device to an aqueous environment.

16. The method of claim 14, wherein 1) 20-30% of the licofelone is released within about 1 hours; 2) 25-65% of the licofelone is released within about 4 hours; 3) 47-83% of the licofelone is released within about 12 hours; and 4 at least 75% of the licofelone is released within ahout 24 hours after exposure of the osmotic device to an aqueous environment.

17. The method of claim 14, wherein the drug-containing coat is present, further comprising an inert water soluble and/or erodible coating disposed between the semipermeable membrane and the drug-containing coating.

18. The method of claim 14, wherein the licofelone is released from the core according to a first order or psuedo-first order rate for a period of at least 12 hours.

19. The method of claim 14, wherein the licofelone is released from the core according to a zero order or psuedo-zero or rate for a period of at least 12 hours.

20. The method of claim 14, wherein the licofelone is released from the core according to a sigmoidal release profile.
